(19) United States
(12) Patent Application Publication
(10) Pub. No.: US 2013/0090504 A1
(43) Pub. Date: Apr. 11, 2013

(54) Methane Enrichment of a Gaseous Alkane Stream for Conversion to Liquid Hydrocarbons

(71) Applicants: John C. Roscoe, Stavanger (NO); John J. Waycuilis, Cypress, TX (US)

(72) Inventors: John C. Roscoe, Stavanger (NO); John J. Waycuilis, Cypress, TX (US)

(21) Appl. No.: 13/269,683

(22) Filed: Oct. 10, 2011

(51) Int. Cl. C07C 1/26

(52) U.S. Cl. 585/310

(57) ABSTRACT

A method is provided for converting gaseous lower molecular weight alkanes contained in a feed gas to liquid higher molecular weight hydrocarbons. One or more lower molecular weight alkanes contained in the feed gas, which are heavier than methane, are converted to methane in a pre-former reactor. The resulting methane-enriched gas has a methane fraction greater than the methane fraction of the feed gas. The methane-enriched gas and bromine are reacted to form alkyl bromide and the alkyl bromide is reacted in the presence of a catalyst to form the liquid higher molecular weight hydrocarbons and a residual gas. The liquid higher molecular weight hydrocarbons are recovered as product and the residual gas is fed to the pre-former reactor with the feed gas.

### BACKGROUND OF THE INVENTION

The present invention relates generally to the conversion of gaseous alkanes to liquid hydrocarbons, and more particularly, to such a conversion process, wherein the methane content of the gaseous alkanes is enriched before conversion to the liquid hydrocarbons.

Natural gas, which is composed of methane and lesser fractions or mere traces of other gaseous alkanes and non-hydrocarbon gases, exists in abundance throughout the world. Natural gas has immediate and direct utility as a relatively low-cost fuel, particularly when produced in proximity to large population centers, thereby minimizing transportation and distribution costs. However, many natural gas reserves are in remote locales far from populated regions. As such, these remote locales lack significant gas pipeline infrastructure or market demand for natural gas. Transportation of natural gas in gaseous form to populated regions where there is a higher market demand, for example, by pipeline or as compressed gas in oceangoing or overland vessels, is relatively expensive. The low density of natural gas imposes practical and economic limits on the distance over which natural gas may be transported in its gaseous state.

Conversion of natural gas from its gaseous state to its liquid state, commonly referred to as liquified natural gas or by its acronym “LNG”, is often performed to more economically transport natural gas over long distances. While transportation of LNG may be less expensive than transportation of natural gas in its gaseous state, LNG conversion processes, such as cryogenic liquefaction, and the corresponding transportation and regasification processes, nevertheless, remain relatively expensive. Furthermore, only a few countries are equipped to import LNG because most lack the requisite regasification facilities.

Aside from fuel, natural gas also has considerable utility as an industrial feedstock for the manufacture of more complex and commercially useful products, such as alcohols, ethers, aldehydes and liquid hydrocarbons made up of C₅₊ alkane, olefin or aromatic constituents which may be useful themselves as fuels or as intermediates in the manufacture of fuels or chemicals, such as lubricants, fuel additives and plastics or other polymers. One technology for commercially manufacturing these products entails converting methane from natural gas to synthesis gas (CO and H₂) at high temperatures. The resulting synthesis gas, commonly referred to as “syngas”, is an intermediate which undergoes synthesis at high pressures to obtain the desired products.

There are several well-known processes for producing synthesis gas from methane. Among them are steam-methane reforming (SMR), partial oxidation (PDX), autothermal reforming (ATR), gas-heated reforming (GHR), and various combinations thereof. As a rule, these processes operate at very high pressures generally up to 100 atmospheres or more and at very high temperatures generally in excess of 600° C. and often in excess of 1000° C. Accordingly, the cost of equipment fabricated from pressure and temperature resistant materials for these high-pressure, high-temperature environments is significant. In addition high-pressure waste-heat boilers are required in these processes at a substantial capital cost to quench and cool the synthesis gas effluent. Significant capital cost and large amounts of power are also required to compress oxygen or air used in these high-pressure, high-temperature processes. Furthermore, production of synthesis gas is generally believed to be thermodynamically and chemically inefficient because it can produce large excesses of waste heat and unwanted carbon dioxide which tend to lower the conversion efficiency of the overall process. Thus, manufacturing technologies using synthesis gas as an intermediate are generally considered unduly expensive resulting in high-cost end products.

In the case where synthesis gas and methanol are intermediates in the manufacture of higher-value downstream products, such as chemical feedstocks or solvents, the cost of producing synthesis gas can render the final end products economically prohibitive. In the case where synthesis gas is an intermediate in the manufacture of liquid hydrocarbons for use as fuels or other petroleum-based products, the cost of producing synthesis gas can likewise render the resulting liquid hydrocarbons economically impractical. The Fischer-Tropsch process is one well-known gas-to-liquids (GTL) technology for converting a synthesis gas intermediate to desirable liquid hydrocarbons. However, the high capital cost for the Fischer-Tropsch process is another economic disincentive to the manufacture of liquid hydrocarbons using a synthesis gas intermediate. In sum, the production of synthesis gas as an industrial manufacturing intermediate represents a large fraction of the capital cost for most known natural gas conversion processes.

An alternate technology to the Fischer-Tropsch process for converting gaseous hydrocarbons to liquid hydrocarbons is taught by U.S. Patent Application Publication No. US 2008/0275284 A1, to Waycuilis, which is incorporated herein by reference. In accordance with this teaching, gaseous alkanes in a combined recycle and natural gas feed stream are contacted with bromine in a bromination reactor at a relatively low temperature to form alkyl bromides. The resulting alkyl bromides are contacted with a selective catalyst in a synthesis reactor at a controlled low temperature to produce desirable liquid hydrocarbons.

The preferred primary alkane reactant in the bromination reactor is methane which is preferably converted therein to mono-bromomethane. The mono-bromomethane is in turn the primary alkyl bromide reactant in the synthesis reactor which is converted therein to desirable liquid hydrocarbons. In addition to methane, however, the natural gas and recycle feed stream to the bromination reactor also typically contains C₂ to C₄ alkane constituents, sometimes in significant quantities.

One feature of the instant invention is the finding that the presence of C₂ to C₄ alkane constituents in the recycle stream to the bromination reactor is problematic. In particular, C₂ to C₄ alkane constituents have been found to be much more reactive with bromine relative to the reactivity of methane with bromine. As a result, the C₂ to C₄ alkane constituents undesirably tend to become poly-brominated when reacted with bromine and also undesirably tend to substantially increase soot production in the bromination reactor relative to bromine. Consequently, the presence of C₂ to C₄ alkane constituents in the recycle stream to the bromination reactor lowers the carbon efficiency of the overall process and increases the bromine requirement for a given amount of alkane conversion. In addition, the formation of soot in the bromination reactor undesirably causes soot deposition in the bromination reactor, increases the required frequency of oxidative regeneration of bromine for the bromination reactor and also accelerates deposition and fouling of the catalyst in the downstream synthesis reactor.

Accordingly, it is believed that the overall efficiency, operability and effectiveness of liquid hydrocarbon production is enhanced by inputting a methane-enriched gaseous reactant stream to the bromination reactor. As such, it is an object of the present invention to increase the methane content of the gaseous input stream to a bromination reactor employed in a process for converting gaseous hydrocarbons to liquid hydrocarbons. The present invention as described hereafter is directed toward achieving these objectives and other objectives which will be apparent to the skilled artisan from the following description.

### SUMMARY OF THE INVENTION

The present invention is a method for converting gaseous lower molecular weight alkanes to liquid higher molecular weight hydrocarbons which comprises converting at least one C₂ to C₅ constituent contained in a feed gas to methane, thereby forming a methane-enriched gas from the feed gas. The methane fraction of the resulting methane-enriched gas is greater than the methane fraction of the feed gas. The methane-enriched gas and bromine are reacted to form alkyl bromide and the alkyl bromide is reacted in the presence of a catalyst to form the liquid higher molecular weight hydrocarbons and a residual gas.

In accordance with one embodiment, the feed gas preferably contains methane and the methane fraction of the feed gas is preferably greater than the C₂ to C₅ fraction of the feed gas. In accordance with another embodiment, the at least one C₂ to C₅ constituent contained in the feed gas is preferably converted to methane by pre-reforming the feed gas and, more particularly, is preferably pre-reformed by contacting the feed gas with a pre-former reactant in the presence of a pre-reforming catalyst. The methane fraction of the resulting methane-enriched gas is preferably greater than about 50 mole % and more preferably at least about 98 mole %. In accordance with yet another embodiment, the feed gas is pre-reformed in a pre-reformer reactor. At least a portion of the residual gas is also preferably fed to the pre-reformer reactor for pre-reforming therein.

The present invention is alternately characterized as a method for converting gaseous lower molecular weight alkanes to liquid higher molecular weight hydrocarbons which comprises separating a C₃+ fraction from a feed gas containing lower molecular weight alkanes to form a C₃+ product and an ethane-rich gas. The ethane fraction of the resulting ethane-rich gas is greater than the ethane fraction of the feed gas. The method further comprises converting at least some of the ethane in the ethane-rich gas to methane, thereby forming a methane-enriched gas from the ethane-rich gas. The methane fraction of the resulting methane-enriched gas is greater than the methane fraction of the ethane-rich gas. The methane-enriched gas and bromine are reacted to form alkyl bromide and the alkyl bromide is reacted in the presence of a catalyst to form the liquid higher molecular weight hydrocarbons and a residual gas.

In accordance with one embodiment, the feed gas preferably contains methane and the methane-enriched gas preferably has a methane fraction of at least about 98 mole %. In accordance with another embodiment, the ethane contained in the ethane-rich gas is preferably converted to methane by pre-reforming the ethane-rich gas and, more particularly, is preferably pre-reformed by contacting the feed gas with a pre-former reactant in the presence of a pre-reforming catalyst. In accordance with yet another embodiment, the ethane-rich gas is pre-reformed in a pre-reformer reactor. In accordance with another embodiment, at least a portion of the residual gas is preferably fed to the pre-reformer reactor. In accordance with still another embodiment, the C₃₊ fraction is separated from the feed gas in a feed gas separator. At least a portion of the residual gas is preferably recycled to the feed gas separator and mixed with the gas therein.

The present invention is alternately characterized as a method for converting gaseous lower molecular weight alkanes to liquid higher molecular weight hydrocarbons which comprises separating a C₂₊ fraction from a feed gas containing lower molecular weight alkanes to form an ethane-rich gas and a first methane-enriched gas. The methane fraction of the first methane-enriched gas is greater than the methane fraction of the feed gas and the ethane fraction of the ethane-rich gas is greater than the ethane fraction of the feed gas. The method further comprises converting at least some the ethane in the ethane-rich gas to methane, thereby forming a second methane-enriched gas having a methane fraction greater than a methane fraction of the ethane-rich gas. The resulting first and second methane-enriched gases and bromine are reacted to form alkyl bromide and the alkyl bromide is reacted in the presence of a catalyst to form the liquid higher molecular weight hydrocarbons and a residual gas.

In accordance with one embodiment, carbon dioxide is separated from the second methane-enriched gas before reacting the second methane-enriched gas and bromine. In accordance with another embodiment, the methane fraction of each of the first and second methane-enriched gases is preferably at least about 98 mole %. In accordance with yet another embodiment, the ethane contained in the ethane-rich gas is converted to methane by pre-reforming the ethane-rich gas. The ethane-rich gas is preferably pre-reformed in a pre-reformer reactor. The ethane-rich gas is preferably pre-reformed by contacting the ethane-rich gas with a pre-former reactant in the presence of a pre-reforming catalyst. The method preferably further comprises feeding at least a portion of the residual gas to the pre-reformer reactor and pre-reforming the residual gas therein. In accordance with still another embodiment, the C₂₊ fraction is separated from the feed gas in a feed gas separator. The method preferably further comprises recycling a portion of the residual gas to the feed gas separator and mixing the residual gas with the gas therein.

The invention will be further understood from the accompanying drawings and description.

### BRIEF DESCRIPTION OF THE DRAWINGS

The accompanying drawings illustrate certain aspects of the present invention, but should not be viewed as by themselves limiting or defining the invention.

FIG. 1 is a block flow diagram of a gaseous alkanes to liquid heavier hydrocarbon products conversion process of the present invention having a gas treatment sequence and a gas conversion sequence;

FIG. 2 is a block flow diagram of another embodiment of the gaseous alkanes to liquid heavier hydrocarbon products conversion process of the present invention having an alternate gas treatment sequence;

FIG. 3 is a block flow diagram of another embodiment of the gaseous alkanes to liquid heavier hydrocarbon products conversion process of the present invention having a different alternate gas treatment sequence;

FIG. 4 is a block flow diagram of another embodiment of the gaseous alkanes to liquid heavier hydrocarbon products conversion process of the present invention having an alternate gas conversion sequence; and

FIG. 5 is a block flow diagram of another embodiment of the gaseous alkanes to liquid heavier hydrocarbon products conversion process of the present invention having a different alternate gas conversion sequence.

### DESCRIPTION OF PREFERRED EMBODIMENTS

Certain embodiments of the process of the present invention are described below. Although aspects of what are to believed to be the primary chemical reactions involved in the present invention are discussed as they are believed to occur, it should be understood that other side reactions may take place. One should not assume that the failure to discuss any particular side reaction herein means that this side reaction does not occur. Conversely, the primary reactions discussed below should not be considered exhaustive or limiting.

As utilized throughout this description, the term “lower molecular weight alkanes” refers generally to alkanes in the range of C₁ to C₅. As such, lower molecular weight alkanes include methane, ethane, propane, butane, pentane, or various mixtures thereof, all of which commonly exist in a gaseous state at ambient atmospheric temperature and pressure. The term “alkyl bromides”, as used in this description, refers generally to mono-, di-, tri-brominated alkanes or mixtures thereof, while use of the specific numerical prefix for the bromine constituent in association with the specific alkane name refers to that respective brominated species only, e.g., “mono-bromomethane” refers to a particular chemical species having a methyl group and a single bromine atom substituted for a carbon atom in the methyl group.

A feed gas for the process of the present invention is preferably a hydrocarbon gas and is more preferably a natural gas as this term is commonly understood in the industry. The composition of a given natural gas may vary somewhat depending on its source. As a rule, however, the bulk of the composition of any natural gas is gaseous hydrocarbons and, more particularly, lower molecular weight alkanes. Of the lower molecular weight alkanes, methane typically makes up the bulk fraction, if not all, of the natural gas composition while ethane typically makes up a smaller, yet nevertheless significant fraction. Butane and propane, if present at all, typically make up still smaller fractions of the lower molecular weight alkanes in the natural gas composition. Pentane, if likewise present at all, typically makes up yet a still smaller fraction.

In addition to gaseous hydrocarbons, a typical natural gas composition also contains one or more gaseous non-hydrocarbon constituents such as nitrogen, carbon dioxide, oxygen, sulfur compounds, and inert gases, e.g., helium, argon, neon and xenon. A representative natural gas composition in accordance with the above-description is listed below in mole % fractions for purposes of illustration, but not limitation: C₁ fraction (methane) 70-90%; C₂ to C₅ fraction (ethane, propane, butane, pentane combined) 0-20%; carbon dioxide 0-8%; oxygen 0-0.2%; nitrogen: 0-5%; hydrogen sulfide: 0-5%; and inert gases (Ar, He, Ne, Xe) trace.

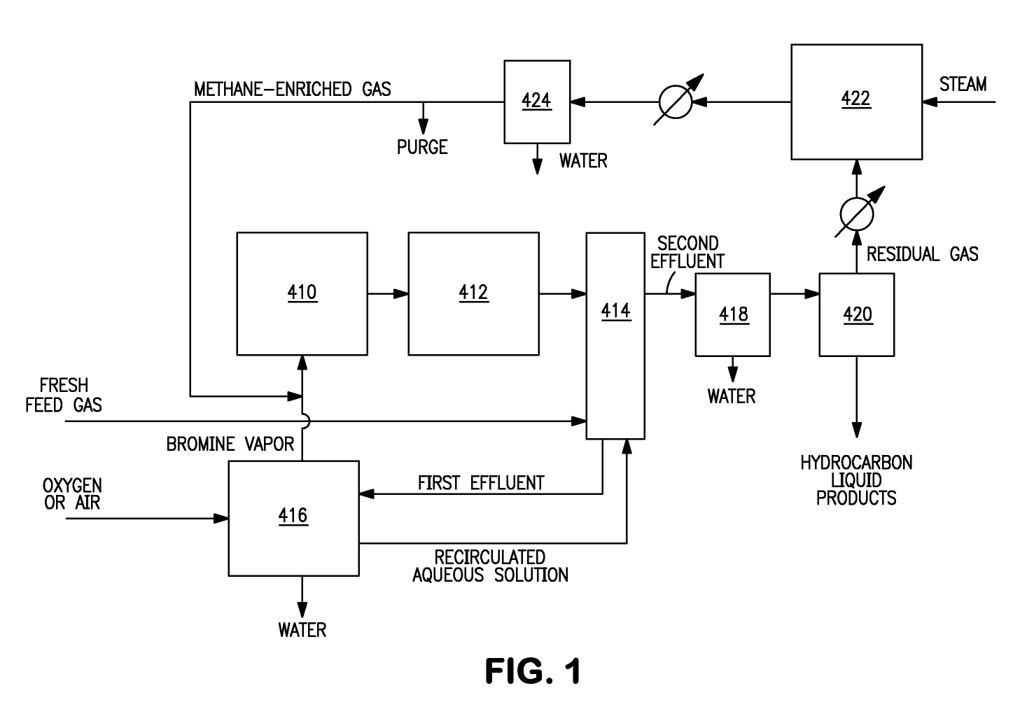

FIG. 1 is a block flow diagram of an embodiment of the process of the present invention. The process is generally depicted as a gas-to-liquids conversion process, wherein gaseous lower molecular weight alkanes are converted to heavier liquid hydrocarbon products by means of multiple process stages. The process stages include an alkane bromination stage 410, an alkyl bromide conversion stage 412, a hydrogen bromide (HBr) separation stage 414, a hydrogen bromide oxidation stage 416, a dehydration stage 418, a liquid product recovery stage 420, a gas enrichment stage 422, and a secondary dehydration stage 424. For purposes of illustration, stages 410, 412, 414, 416, 418, 420 are conceptually grouped together and termed a gas conversion sequence, while remaining stages 422, 424 are conceptually grouped together and termed a gas treatment sequence.

The present process is practiced by initiating the gas conversion sequence, wherein a methane-enriched gas is combined with bromine vapor. The methane-enriched gas and bromine vapor react in the alkane bromination stage 410 to form a reaction product which includes gaseous alkyl bromides and hydrogen bromide vapor. The effluent from the alkane bromination stage 410, which contains the reaction product, is fed to the alkyl bromide conversion stage 412 and the gaseous alkyl bromides are reacted therein to form higher molecular weight hydrocarbons and additional hydrogen bromide vapor.

The resulting effluent from the alkyl bromide conversion stage 412, which includes the higher molecular weight hydrocarbons and hydrogen bromide vapor, is fed to the hydrogen bromide separation stage 414 where it is contacted with a recirculated aqueous solution likewise fed to the hydrogen bromide separation stage 414. The hydrogen bromide vapor dissolves in the recirculated aqueous solution and the resulting solution is discharged from the hydrogen bromide separation stage 414 as a first effluent thereof and fed to the hydrogen bromide oxidation stage 416.

The dissolved hydrogen bromide vapor in the first effluent is either in the form of hydrobromic acid or a metal bromide salt depending on whether the recirculated aqueous solution has neutralized the hydrobromic acid in the hydrogen bromide separation stage 414 or not. If not, the hydrobromic acid is neutralized in the hydrogen bromide oxidation stage 416 to form the metal bromide salt. In either case, the metal bromide salt is oxidized to form elemental bromine in the hydrogen bromide oxidation stage 416 by contact with oxygen or air, which is also supplied to the hydrogen bromide oxidation stage 416. The resulting elemental bromine is separated and returned to the alkane bromination stage 410 as the bromine vapor to repeat another cycle of bromine utilization within the present process. Excess water formed in the hydrogen bromide oxidation stage 416 is discharged therefrom and the remaining recirculated aqueous solution, which is relatively free of elemental bromine or other bromine constituents, is recirculated back to the hydrogen bromide separation stage 414.

In addition to receiving the alkyl bromide conversion stage effluent and recirculated aqueous solution from the hydrogen bromide oxidation stage 416, the hydrogen bromide separation stage 414 also receives a fresh feed gas. The feed gas contacts the alkyl bromide conversion stage effluent in the hydrogen bromide separation stage 414 to strip the higher molecular weight hydrocarbons therefrom. The resulting mixture, which includes the feed gas and higher molecular weight hydrocarbons, is discharged as a second effluent from the hydrogen bromide separation stage 414 and fed to the dehydration stage 418. Water is separated from the second effluent in the dehydration stage 418 and discharged therefrom. The resulting dehydrated effluent is fed to the product recovery stage 420 where it is divided by gas-liquid separation into a gas stream and a liquid stream. The liquid stream comprises liquid higher molecular weight hydrocarbons which are recovered from the product recovery stage 420 as desirable primary end products, namely hydrocarbon liquid products, thereby completing the gas conversion sequence of the present process.

The gas stream of the product recovery stage 420 is a residual gas comprising the feed gas and essentially any other residual process gases remaining in the product recovery stage 420 after the gas-liquid separation. The residual gas is specifically characterized as containing C₂₊ components. The gas treatment sequence of the process is initiated at this point by cooling the residual gas and feeding it to the gas enrichment stage 422 where the cooled residual gas is contacted with steam, also fed to the gas enrichment stage 422. The steam converts at least some if not substantially all of the C₂₊ components in the residual gas to methane. The effluent from the gas enrichment stage 422 is cooled to condense excess steam and fed to a secondary dehydration stage 424 where the resulting water is separated therefrom and discharged. The remaining gas is characterized as a dehydrated methane-enriched gas. The methane-enriched gas is fed to the alkane bromination stage 410 for use in the gas conversion sequence as describe above, thereby completing the gas treatment sequence of the present process.

It is noted that a first fraction of the methane-enriched gas may optionally be withdrawn from the secondary dehydration stage 424 as a purge stream before the methane-enriched gas is fed to the alkane bromination stage 410. The purge stream inhibits the accumulation of inert gases, such as CO₂ and N₂, in the methane-enriched gas stream and the purge gas in the purge stream may also be used as a fuel to provide process heat. In this case, only the remaining second fraction of the methane-enriched gas from the secondary dehydration stage 424 is fed to the alkane bromination stage 410 after diverting the first fraction of the methane-enriched gas to the purge stream.

In accordance with one preferred embodiment of the gas treatment sequence, the gas enrichment stage 422 comprises a pre-reforming reactor which converts the C₂₊ components of the residual gas discharged from the product recovery stage 420 to methane. In accordance with this preferred embodiment, the feed gas, which is included in the residual gas, is characterized as comprising methane and one or more C₂₊ constituents selected from ethane, propane, butane and/or pentane. The feed gas is specifically characterized as having a C₁ fraction (i.e., methane fraction) which is a majority fraction of the feed gas (i.e., greater than 50%) based on the sum of all the hydrocarbon constituents in the feed gas. The feed gas is further characterized as having a lesser, but significant, C₂ fraction (i.e., ethane fraction). The feed gas may still further be characterized as having lesser or trace fractions of gaseous non-hydrocarbon constituents relative to the methane fraction based on the sum of all the gaseous constituents in the feed gas. The fractions of gaseous non-hydrocarbon constituents may be more specifically characterized as consistent with the above-recited representative natural gas composition with respect to N₂, CO₂, H₂S, etc. In any case, at least some and preferably most or essentially all of the C₂₊ constituents undergo pre-reforming within the pre-reforming reactor of the gas enrichment stage 422 while the non-hydrocarbon constituents preferably pass through the pre-reforming reactor unreacted and preferably have no substantial impact on the gas conversion sequence of the process.

The above-described characteristics of a preferred feed gas are consistent with most natural gases. Nevertheless, although natural gas is a preferred feed gas of the present process, this preference should not necessarily be construed as limiting or defining the scope of the present invention. It is understood that the present invention is alternatively applicable to feed gases other than natural gas which are consistent with the above-described feed gas characteristics. Regardless, as noted above, a fundamental characteristic of a preferred feed gas of the present invention is that it has some fraction of C₂₊ constituents (i.e., contains at least one gaseous alkane constituent heavier than methane) which is subject to pre-reforming. It is also a fundamental characteristic of a preferred feed gas that it has a significant C₂ fraction (i.e., a significant ethane concentration) because ethane is oftentimes a preferred object of pre-reforming as described in greater detail below.

Pre-reforming is generally defined herein as the process of converting gaseous hydrocarbons heavier than methane contained within a feed gas into methane in a resulting product gas which is termed a pre-reformate gas. As such, pre-reforming results in an increase in the overall H/C ratio of the hydrocarbon portion of the product gas relative to the hydrocarbon portion of the feed gas. Stated another way, pre-reforming minimizes the C₂₊ hydrocarbon content of the product gas while maximizing the CH₄ content. A preferred feed for a pre-reforming process is natural gas, which typically has an H/C ratio of about 3.83. As such, a desired pre-reformate gas has an H/C ratio of 4.0, i.e., the only hydrocarbon constituents remaining in the resulting pre-reformate gas are essentially pure methane which is defined herein as a gas which is about 98 mole % methane or greater and is preferably about 99 mole % methane or greater. Pre-reforming is distinguishable from reforming in that the goal of reforming a hydrocarbon feed gas is to convert all gaseous hydrocarbon constituents therein to hydrogen (H₂) in the product gas, whereas the goal of pre-reforming a hydrocarbon feed gas is convert all C₂₊ constituents therein to methane in the pre-reformate gas.

Pre-reforming is carried out by forming a mixture of a hydrocarbon gas, which includes at least one gaseous alkane having a molecular weight greater than methane, and a pre-former reactant such as steam, CO₂, purified O₂ or air. The mixture is heated to an elevated temperature in the presence of a conventional pre-reforming catalyst which is capable of converting at least a portion and preferably most or essentially all of the alkane having a molecular weight greater than methane in the hydrocarbon/reactant mixture to methane.

Although the present invention is not limited to any one specific pre-reforming process or mechanism, a series of reactions for the formation of methane are recited below which define, by way of example, a generalized pre-reforming process having utility in the process of the present invention.

### Higher Hydrocarbon-Steam Reforming:

CₙHₘ+nH₂O→nCO+(n+m/2)H₂,where 2≦n≦4 (1)

### Steam Methane Reforming:

CH₄+H₂O [symbol] CO+3H₂ (2)

### Shift Reaction:

CO+H₂O [symbol] CO₂+H₂ (3)

### Methanation:

CO+3H₂ [symbol] CH₄+H₂O (4)

In accordance with the pre-reforming process defined by the above-recited equations, the hydrocarbon feed CₙHₘ is mixed with superheated high-pressure steam. The resulting feed mixture is preheated and introduced into the pre-reformer reactor where it flows over a bed of pre-reforming catalyst. The catalyst causes adiabatic reforming of the hydrocarbon feed at the temperature within the pre-reforming reactor which produces a pre-reformate gas. The pre-reformate gas contains mostly, if not essentially all, methane, a very low or even negligible C₂₊ fraction, and equilibrium levels of hydrogen, carbon oxides and steam. The pre-reforming catalyst can be selected from among those well-known to one of ordinary skill in the art as having utility in the above-recited pre-reforming process.

The reactions taking place over the catalyst bed are the hydrocarbon reforming reactions (1) and (2) and the shift reaction (3). Reactions (1) and (2) are endothermic as shown from left to right and reactions (3) and (4) are exothermic. Reactions (2), (3) and (4) are reversible. Therefore, once the heavier hydrocarbons are reformed in reaction (1), the carbon monoxide and hydrogen produced thereby react together through the reverse of reaction (2) which is the highly exothermic methanation reaction (4). Reaction (1) is also reversible, but the reaction conditions within the pre-reformer reactor are selected such that the equilibrium value lies very far to the right and hence virtually all heavier hydrocarbons are converted.

When detailed for each C₂ to C₅ alkane, reactions (1)-(4) lead to the following overall chemical reactions:

### Ethane:

4C₂H₆+9H₂O→21H₂+7CO+CO₂→7CH₄+7H₂O+CO₂ (5)

### Propane:

2C₃H₈+7H₂O→15H₂+5CO+CO₂→5CH₄+5H₂O+CO₂ (6)

### Butane:

4C₄H₁₀+19H₂O→39H₂+13CO+3CO₂→13CH₄+13H₂O+3CO₂ (7)

### Pentane:

C₅H₁₂+6H₂O→12H₂+4CO+CO₂→4CH₄+4H₂O+CO₂ (8)

In any case, the output of the pre-reforming reactor in the present embodiment corresponds to the effluent of the gas enrichment stage 422 generally described above, which is fed to the secondary dehydration stage 424. Heat generated in the gas conversion sequence of the present process may also be conveyed to the gas enrichment stage 422 to drive the endothermic reactions occurring therein.

Although not shown in FIG. 1, it is understood that additional gas pre- or post-treatment stages relative to the pre-reformer reactor may be included in the gas treatment sequence. Any additional gas pretreatment stages are preferably positioned upstream of the pre-reformer reactor in the gas enrichment stage 422. Such additional gas pretreatment stages may include heating, cooling, expanding, compressing, concentrating, diluting, drying, or introducing additives to the feed gas alone upstream of the hydrogen bromide separation stage 414 or to the residual gas downstream of the hydrogen bromide separation stage 414. Gas pretreatment may also include removing undesirable gas constituents from the residual gas or the feed gas, such as, H₂S, which may diminish the effectiveness of the pre-reformer reactor.

An exemplary gas pretreatment stage is a hydrodesulfurization (HDS) unit positioned upstream of the pre-reformer reactor to prevent or otherwise diminish sulfur constituents in the residual gas from entering the pre-reformer reactor. In addition, the residual gas or the feed gas alone can be pretreated to saturate any unsaturated hydrocarbons present therein. In any case, the appropriate selection of such additional pretreatment stages, if any, and the manner of performing them are within the purview of one of ordinary skill in the art and are within the scope of the present invention.

Any additional gas post-treatment stages are preferably positioned downstream of the pre-reformer reactor, but upstream of the alkane bromination stage 410 to modify any properties of the feed to the gas conversion sequence which could enhance its effectiveness. For example, it is desirable that no more than about 2 mole % CO₂ be present in the gas conversion sequence in order to limit the necessary purge rate of the methane-enriched gas in the purge stream to about 5 volume % of the total volume of the methane-enriched gas exiting the secondary dehydration stage 424. Accordingly, it may be desirable to include CO₂ removal as a gas post-treatment stage at the end of the gas treatment sequence.

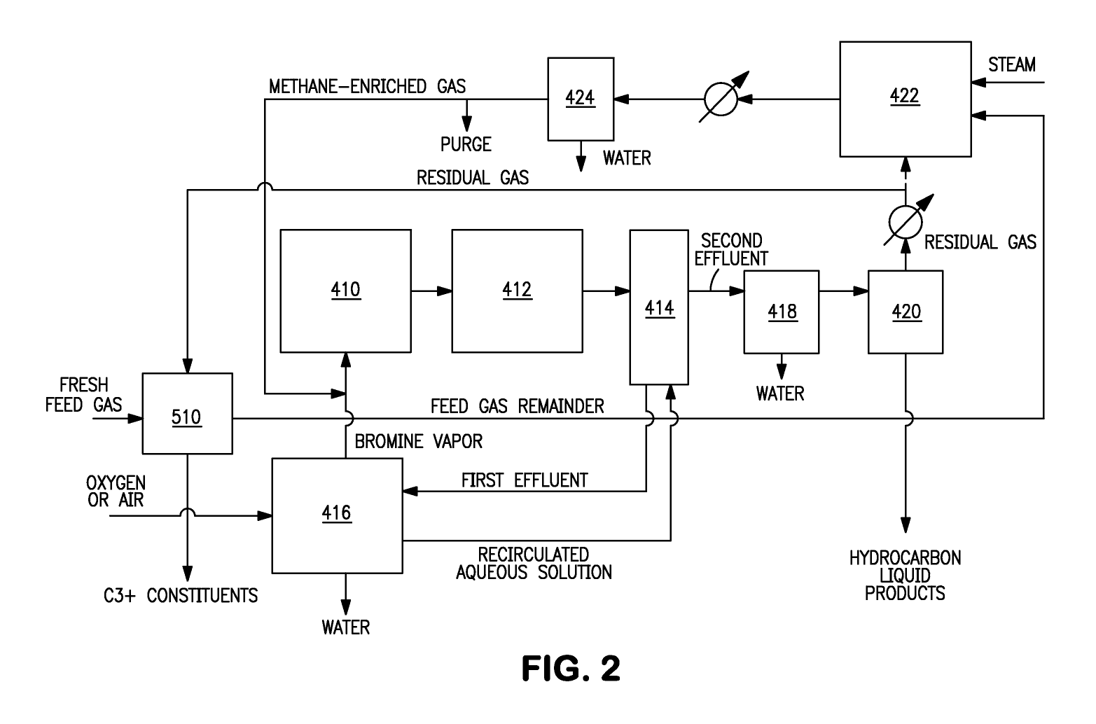

FIG. 2 illustrates an alternate specific embodiment of a gas treatment sequence having utility in the process of the present invention. The embodiment of FIG. 2 differs from the embodiment of FIG. 1 by adding a gas treatment stage 510 to the gas treatment sequence as well as corresponding flow lines. The gas treatment stage 510 is preferably a gas separation and recovery unit for separating out C₃₊ constituents (i.e., propane and/or heavier gaseous alkane constituents) in the feed gas upstream of the gas enrichment stage 422 and downstream of the hydrogen bromide separation stage 414. The remaining elements shown in the embodiment of FIG. 2 which are common to the embodiment of FIG. 1 are designated in FIG. 2 by the same reference characters as used in FIG. 1.

In accordance with the embodiment of FIG. 2, a fresh feed gas having substantially the same properties as the feed gas of the embodiment of FIG. 1 described above is fed to the gas treatment stage 510. At least some and preferably most or essentially all of the C₃₊ constituents in the feed gas are separated therefrom within the gas treatment stage 510 in accordance with any conventional separation technique known to one of ordinary skill in the art. The resulting C₃₊ constituents separated from the feed gas are recovered from the gas treatment stage 510. The recovered C₃₊ constituents typically have value as separate end products of the present process with beneficial uses apart from the primary end products which are the heavier hydrocarbon liquid products obtained in the gas conversion sequence. As such, the recovered C₃₊ constituents are removed from the process flow path as desirable secondary end products.

The feed gas remainder in the gas treatment stage 510 is termed an ethane-rich gas because the ethane fraction in the feed gas remainder is greater than the ethane fraction in the fresh feed gas entering the gas treatment stage 510. Unlike the embodiment of FIG. 1, the feed gas remainder bypasses stages 414, 418 and 420 of the gas conversion sequence and is fed directly to the gas enrichment stage 422 of the gas treatment sequence. At least some and preferably most or essentially all of the ethane in the ethane-rich gas undergoes pre-reforming within the pre-reforming reactor of the gas enrichment stage 422 in substantially the same manner as described above with respect to the embodiment of FIG. 1. In the event any C₃₊ constituents remain in the ethane-rich gas after the gas treatment stage 510, these constituents likewise preferably undergo pre-reforming within the pre-reforming reactor of the gas enrichment stage 422.

The effluent of the pre-reforming reactor is a mixture having a composition substantially similar to that of the embodiment of FIG. 1. The effluent is conveyed to the secondary dehydration stage 424 where the water is separated and removed from the process stream leaving the methane-enriched gas. As in the embodiment of FIG. 1, a fraction of the methane-enriched gas from the secondary dehydration stage 424 may optionally be withdrawn as a purge stream, which may be used as a fuel to provide process heat. All of the methane-enriched gas, or the remaining fraction if a purge stream is withdrawn, is conveyed to the alkane bromination stage 410 to initiate the gas conversion sequence, which is substantially similar to the gas conversion sequence in the embodiment of FIG. 1.

In accordance with the present embodiment, all or a fraction of the residual gas remaining after gas-liquid separation in the product recovery stage 420 is recycled directly to the gas treatment stage 510. If only a fraction of the residual gas from the product recovery stage 420 is recycled to the gas treatment stage 510, the remainder is fed to the pre-reforming reactor of the gas enrichment stage 422 where it undergoes pre-reforming in substantially the same manner as described above.

As in the embodiment of FIG. 1, additional gas pretreatment or post-treatment stages may be included in the gas treatment sequence of FIG. 2 which are not otherwise shown therein. Likewise, heat generated in the gas conversion sequence of FIG. 2 may be utilized in the gas treatment sequence.

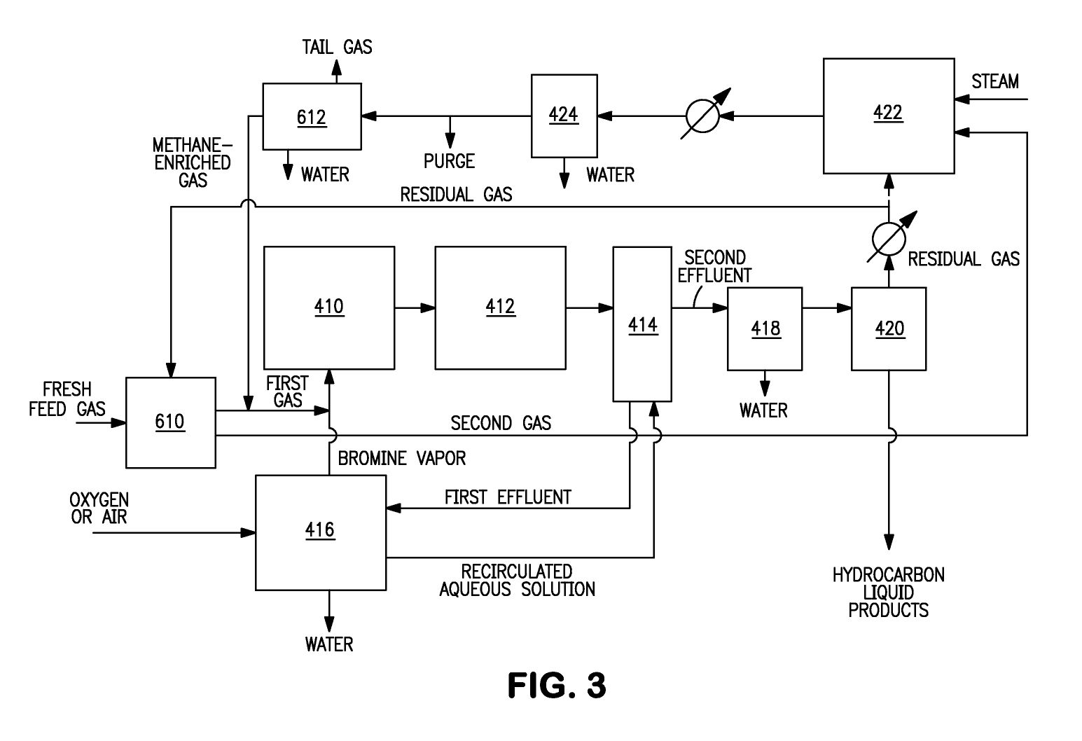

FIG. 3 illustrates another alternate embodiment of a gas treatment sequence having utility in the process of the present invention. The embodiment of FIG. 3 differs from the embodiment of FIG. 2 by substituting a first gas treatment stage 610 for the gas treatment stage 510 in the gas treatment sequence. The embodiment of FIG. 3 further differs from the embodiment of FIG. 2 by adding a second gas treatment stage 612 to the gas treatment sequence as well as corresponding flow lines. The remaining elements shown in the embodiment of FIG. 3 which are common to the embodiment of FIG. 1 are designated in FIG. 3 by the same reference characters as used in FIG. 1.

In accordance with the embodiment of FIG. 3, a fresh feed gas having substantially the same properties as the feed gas of the embodiments of FIGS. 1 and 2 described above is introduced into the first gas treatment stage 610 which is preferably a gas separation unit for separating out the C₂₊ constituents in the feed gas. At least some and preferably most or essentially all of the C₂₊ constituents as well as any non-hydrocarbon constituents (to the extent they are present) are separated from the feed gas within the first gas treatment stage 610 in accordance with any conventional separation technique known to one of ordinary skill in the art. As such the first gas treatment stage 610 splits the initial fresh feed gas into two separate gas streams, namely a first gas stream and a second gas stream. The first gas stream contains a first gas which is the remainder of the feed gas after the bulk of the C₂₊ hydrocarbon constituents and non-hydrocarbon constituents have been separated out by the first gas treatment stage 610. The first gas is termed a first methane-enriched gas because the methane fraction of the first gas is greater than the methane fraction of the initial fresh feed gas. The first gas preferably has a methane fraction of about 98 mole % or greater and more preferably about 99 mole % or greater.

The second gas stream contains a second gas which is the bulk of the C₂₊ constituents and non-hydrocarbon constituents from the initial fresh feed gas that have been separated out by the first gas treatment stage 610. The second gas is termed an ethane-rich gas because the ethane fraction of the second gas is greater than the ethane fraction of the initial fresh feed gas. The second gas is conveyed to the pre-reforming reactor of the gas enrichment stage 422. At least some and preferably most or essentially all of the ethane in the second gas undergoes pre-reforming within the pre-reforming reactor in substantially the same manner as described above with respect to the embodiments of FIGS. 1 and 2. Any C₃₊ constituents in the second gas likewise preferably undergo pre-reforming within the pre-reforming reactor.

The product of the pre-reforming reactor is a product mixture having a composition similar to the output of the pre-reforming reactor in the embodiments of FIGS. 1 and 2. The product mixture is conveyed to the secondary dehydration unit 424 where the water is separated and removed from the process stream leaving a second methane-enriched gas. In a similar manner to the embodiments of FIGS. 1 and 2, a fraction of the resulting second methane-enriched gas from the secondary dehydration stage 424 may optionally be withdrawn as a purge stream, which may be used as a fuel to provide process heat. All of the second methane-enriched gas, or the remaining fraction if a purge stream is withdrawn, is conveyed to the second gas treatment stage 612, which is preferably a gas separation unit for removal of the non-hydrocarbon constituents in the second methane-enriched gas input thereto. As such, at least some and preferably most or essentially all of the non-hydrocarbon constituents in the second methane-enriched gas are separated therefrom within the second gas treatment stage 612 in accordance with any conventional separation technique known to one of ordinary skill in the art.

The resulting output of the second gas treatment stage 612 is a first and a second gas output stream and an aqueous output stream. The first gas output stream is a tail gas which comprises the non-hydrocarbon constituents, such as carbon dioxide, and which may also comprise a small residual amount of gaseous hydrocarbons. The tail gas may be treated further as necessary so that it is suitable to be burned as a fuel which provides a secondary heat source for the present process or for some unrelated process not shown. Alternatively, the tail gas may be otherwise disposed in an environmentally compatible manner. The second gas output stream is the second methane-enriched gas having the non-hydrocarbon constituents removed therefrom and having a methane fraction preferably about 98 mole % or greater and more preferably about 99 mole % or greater. The aqueous output stream comprises any water which is removed from the second methane-enriched gas by additional dehydration of the input gas in the second gas treatment stage 612.

The first gas, i.e., the first methane-enriched gas, output from the first gas treatment stage 610 is combined with the second methane-enriched gas output from the second gas treatment stage 612 to form a gas mixture, termed a combined methane-enriched gas. The combined methane-enriched gas is fed to the alkane bromination stage 410 to initiate the gas conversion sequence, which is substantially similar to the gas conversion sequences in the embodiments of FIGS. 1 and 2. As in the embodiments of FIGS. 1 and 2, additional gas pretreatment or post-treatment stages may be included in the gas treatment sequence of FIG. 3 which are not otherwise shown therein. Likewise, heat generated in the gas conversion sequence of FIG. 3 may be utilized in the gas treatment sequence.

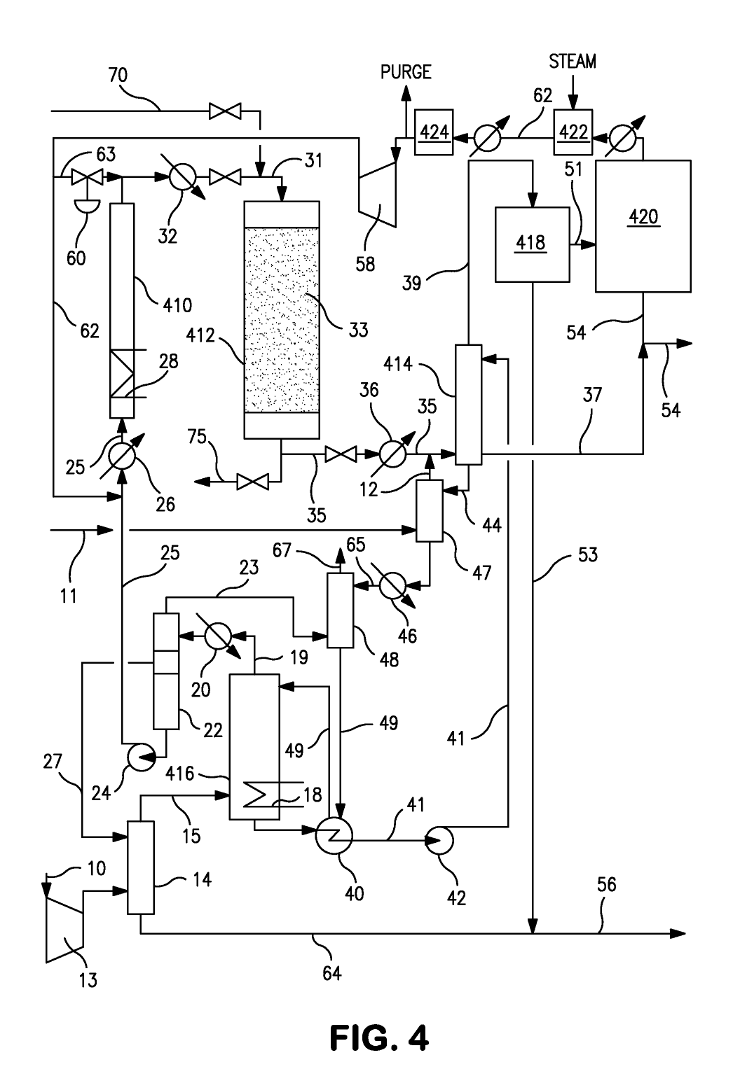

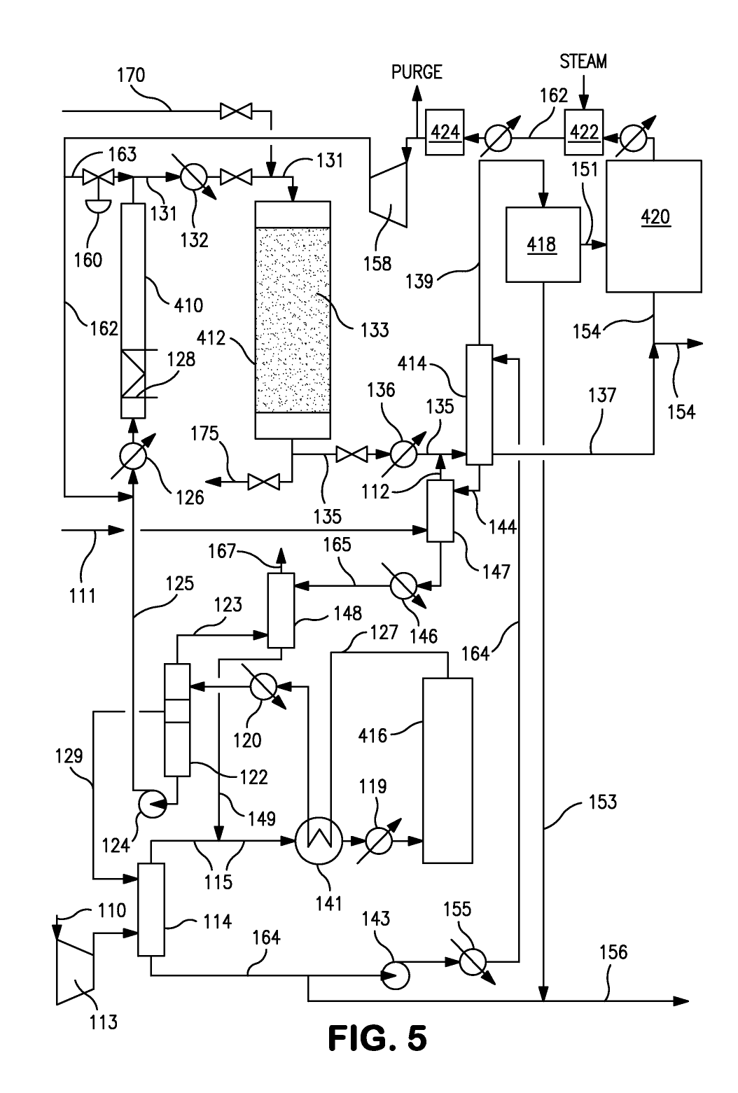

The gas conversion sequence is described only generally above in cooperation with specific embodiments of the gas treatment sequence. However, specific embodiments of the gas conversion sequence are described in detail below with reference to FIGS. 4 and 5. These embodiments of the gas conversion sequence are intended to be integrated with the specific embodiments of the gas treatment sequence described in detail above with reference to FIGS. 1-3 to fully characterize alternate embodiments of the gas-to-liquids conversion process of the present invention.

Referring to FIG. 4, an embodiment of the gas-to-liquids conversion process of the present invention is shown wherein a gas treatment sequence of FIG. 1 is integrated with a specific embodiment of a gas conversion sequence. As such, line 11 of FIG. 4 and associated process units correspond to the feed gas input in FIG. 1 and line 62 of FIG. 4 and associated process units correspond to the gas treatment sequence in FIG. 1. Furthermore, process units 30, 34, 38, 16, 50 and 52 of FIG. 4 correspond to stages 410, 412, 414, 416 and 418, respectively, of FIG. 1. The remaining elements shown in the embodiment of FIG. 4 which are common to the embodiment of FIG. 1 are designated in FIG. 4 by the same reference characters as used in FIG. 1.

In accordance with the embodiment of FIG. 4, a fresh feed gas having a substantially similar composition as the feed gas described above with reference to FIG. 1 is directed via line 11 to a hydrocarbon stripper 47 described in greater detail hereafter. A methane-enriched gas likewise having a substantially similar composition as the methane-enriched gas described above with reference to FIG. 1 exits the compressor 58 in line 62 downstream of the secondary dehydration stage 424 at a pressure in the range of about 1 bar to about 30 bar and is directed into line 25 where it mixes with dry liquid bromine being transported via line 25 by pump 24. The methane-enriched gas and dry liquid bromine pass through heat exchanger 26 wherein the liquid bromine is vaporized to dry bromine vapor. The resulting mixture is fed to a first reactor 30 (i.e., the alkane bromination stage). The molar ratio of lower molecular weight alkanes to dry bromine vapor in the mixture introduced into first reactor 30 is preferably in excess of 2.5:1. First reactor 30 has an inlet pre-heater zone 28 which heats the mixture to a reaction initiation temperature in the range of about 250° C. to about 400° C.

The lower molecular weight alkanes react exothermically with the dry bromine vapor in first reactor 30 at a relatively low temperature in the range of about 250° C. to about 600° C. and at a pressure in the range of about 1 bar to about 30 bar to produce gaseous alkyl bromides and hydrogen bromide vapors. The upper limit of the operating temperature range is greater than the upper limit of the reaction initiation temperature range to which the feed mixture is heated due to the exothermic nature of the bromination reaction. In the case where the lower molecular weight alkane is methane, methyl bromide is formed in accordance with the following general reaction:

CH₄(g)+Br₂(g)→CH₃Br(g)+HBr(g)

This reaction occurs with a significantly high degree of selectivity to methyl bromide. Furthermore, selectivity to the mono-halogenated methyl bromide, i.e., mono-bromomethane, increases using a methane to bromine ratio of about 4.5:1 relative to the selectivity obtained using smaller methane to bromine ratios. Small amounts of dibromomethane and tribromomethane are also formed in the bromination reaction. Higher alkanes, such as ethane, propane and butane, although preferably present at most only in negligible amounts, are also readily brominated resulting in mono and multiple brominated species such as ethyl bromides, propyl bromides and butyl bromides. If an alkane to bromine ratio of significantly less than about 2.5 to 1 is utilized, a lower selectivity to methyl bromide occurs and significant formation of undesirable carbon soot is observed.

The dry bromine vapor that is fed into first reactor 30 is substantially water-free. It has been discovered that elimination of substantially all water vapor from the bromination step in first reactor 30 substantially eliminates the formation of unwanted carbon dioxide, thereby increasing the selectivity of alkane bromination to alkyl bromides and eliminating the large amount of waste heat generated in the formation of carbon dioxide from alkanes.

The effluent from first reactor 30, which contains alkyl bromides and hydrogen bromide, is withdrawn via line 31 and partially cooled in heat exchanger 32 before being conveyed to a second reactor 34. The temperature to which the effluent is partially cooled in heat exchanger 32 is in the range of about 150° C. to about 350° C. when it is desired to convert the alkyl bromides to higher molecular weight hydrocarbons in a second reactor 34 (i.e., the alkyl bromide conversion stage) or in the range of about 150° C. to about 450° C. when it is desired to convert the alkyl bromides to olefins in second reactor 34. The alkyl bromides are reacted exothermically in second reactor 34 over a fixed bed 33 of crystalline alumino-silicate catalyst. The temperature and pressure employed in second reactor 34 as well as the specific crystalline alumino-silicate catalyst determine the actual product(s) formed in second reactor 34. The crystalline alumino-silicate catalyst in fixed bed 33 is preferably a zeolite catalyst and most preferably a ZSM-5 zeolite catalyst when it is desired to form higher molecular weight hydrocarbons. Although the zeolite catalyst is preferably in the hydrogen, sodium or magnesium form, the zeolite may also be modified by ion exchange with other alkali metal cations, such as Li, Na, K or Cs, with alkali-earth metal cations, such as Mg, Ca, Sr or Ba, or with transition metal cations, such as Ni, Mn, V, W, or to the hydrogen form. Other zeolite catalysts having varying pore sizes and acidities, which are synthesized by varying the alumina-to-silica ratio may be used in second reactor 34 as will be evident to a skilled artisan.

When it is desired to form olefins in second reactor 34, the crystalline alumino-silicate catalyst in fixed bed 33 is preferably a zeolite catalyst and most preferably an X type or Y type zeolite catalyst. A preferred zeolite is 10X or Y type zeolite, although other zeolites with differing pore sizes and acidities, which are synthesized by varying the alumina-to-silica ratio may be used in the process as will be evident to a skilled artisan. Although the zeolite catalyst is preferably used in a protonic form, a sodium form or a mixed protonic/sodium form, the zeolite may also be modified by ion exchange with other alkali metal cations, such as Li, K or Cs, with alkali-earth metal cations, such as Mg, Ca, Sr or Ba, or with transition metal cations, such as Ni, Mn, V, W, or to the hydrogen form. These various alternative cations have an effect of shifting reaction selectivity. Other zeolite catalysts having varying pore sizes and acidities, which are synthesized by varying the alumina-to-silica ratio, may be used in second reactor 34 as will be evident to a skilled artisan.

The temperature at which second reactor 34 is operated is an important parameter in determining the selectivity of the reaction to higher molecular weight hydrocarbons or to olefins. Where a catalyst is selected to form higher molecular weight hydrocarbons in second reactor 34, it is preferred to operate second reactor 34 at a temperature within the range of about 150° to 450° C. Temperatures above about 300° C. in second reactor 34 result in increased yields of light hydrocarbons, such as undesirable methane, whereas lower temperatures increase yields of higher molecular weight hydrocarbon products. At the low end of the temperature range, for example, with methyl bromide reacting over ZSM-5 zeolite at temperatures as low as 150° C., methyl bromide conversion on the order of 20% is noted with a high selectivity toward C₅+ products. When the alkyl bromide reaction is carried out over the preferred zeolite ZSM-5 catalyst, cyclization reactions also occur such that C₇+ fractions are composed primarily of substituted aromatics.

At increasing temperatures approaching 300° C., methyl bromide conversion increases towards 90% or greater. However, selectivity towards C₅+ products decreases and selectivity towards lighter products, particularly undesirable methane, increases. Surprisingly very little ethane or C₂-C₃ olefin constituents are formed. At temperatures approaching 450° C. almost complete conversion of methyl bromide to methane occurs.

In the optimum operating temperature range between about 300° C. and 400° C., a small amount of carbon will build up on the catalyst over time during operation as a byproduct of the reaction, which causes a decline in catalyst activity over a range of hours, up to hundreds of hours, depending on the reaction conditions and the composition of the inlet gas. It is believed that higher reaction temperatures above about 400° C. associated with the formation of methane favor the thermal cracking of alkyl bromides and formation of carbon or coke and, hence, an increase in the rate of deactivation of the catalyst. Conversely, temperatures at the lower end of the range, particularly below about 300° C., may also contribute to coking due to a reduced rate of desorption of heavier products from the catalyst. Hence, operating temperatures within the range of about 150° C. to about 450° C., but preferably in the range of about 300° C. to about 400° C. in second reactor 34 balance increased selectivity of the desired C₅+ products and lower rates of deactivation due to carbon formation against higher conversion per pass, which minimizes the quantity of catalyst, recycle rates and equipment size required.

Where a catalyst is selected to form olefins in second reactor 34, it is preferred to operate second reactor 34 at a temperature within the range of about 250° C. to 500° C. Temperatures above about 450° C. in second reactor 34 can result in increased yields of light hydrocarbons, such as undesirable methane, and also deposition of coke, whereas lower temperatures increase yields of ethylene, propylene, butylene and higher molecularweight hydrocarbon products. When the alkyl bromide reaction is carried out over the preferred 10X zeolite catalyst, it is believed that cyclization reactions also occur such that C₇+ fractions contain substantial substituted aromatics.

At increasing temperatures approaching 400° C., it is believed that methyl bromide conversion increases towards 90% or greater. However, selectivity towards C₅+ products decreases and selectivity towards lighter products, particularly olefins, increases. At temperatures exceeding 550° C., it is believed that a high conversion of methyl bromide to methane and carbonaceous coke occurs.

In the preferred operating temperature range between about 300° C. and 450° C., a lesser amount of coke will likely build up on the catalyst over time during operation as a byproduct of the reaction. It is believed that higher reaction temperatures above about 400° C., associated with the formation of methane, favor the thermal cracking of alkyl bromides and formation of carbon or coke and, hence, an increase in the rate of deactivation of the catalyst. Conversely, temperatures at the lower end of the range, particularly below about 300° C., may also contribute to coking due to a reduced rate of desorption of heavier products from the catalyst. Hence, operating temperatures within the range of about 250° C. to about 500° C. in second reactor 34, but preferably in the range of about 300° C. to about 450° C. balance increased selectivity of the desired olefins and C₅+ products and lower rates of deactivation due to carbon formation against higher conversion per pass, which minimizes the quantity of catalyst, recycle rates and equipment size required.

The catalyst may be periodically regenerated in situ by isolating second reactor 34 from the normal process flow. Once isolated, second reactor 34 is purged with an inert gas via line 70 at a pressure in a range from about 1 to about 5 bar at an elevated temperature in the range of about 400° C. to about 650° C. to remove unreacted material adsorbed on the catalyst insofar as is practical. The deposited carbon is subsequently oxidized to CO₂ by addition of air or inert gas-diluted oxygen to second reactor 34 via line 70 at a pressure in the range of about 1 bar to about 5 bar at an elevated temperature in the range of about 400° C. to about 650° C. Carbon dioxide and residual air or inert gas are vented from second reactor 34 via line 75 during the regeneration period.

The effluent from second reactor 34, which comprises hydrogen bromide and higher molecular weight hydrocarbons, olefins or mixtures thereof, is withdrawn via line 35 and cooled to a temperature in the range of 0° C. to about 100° C. in exchanger 36. The cooled effluent in line 35 is combined with vapor effluent in line 12 from hydrocarbon stripper 47, which contains the methane-enriched gas and residual higher molecular weight hydrocarbons stripped-out by contact with the methane-enriched gas in hydrocarbon stripper 47. The resulting combined vapor mixture is passed to a scrubber 38 (i.e., the hydrogen bromide separation stage) and contacted with a concentrated aqueous partially-oxidized metal bromide salt solution, which is transported to scrubber 38 via line 41.

The concentrated aqueous partially-oxidized metal bromide salt solution contains metal hydroxide, metal oxide, metal oxy-bromide or mixtures of these species. The preferred metal of the bromide salt is Fe(III), Cu(II) or Zn(II), or mixtures thereof, which are less expensive and readily oxidize at lower temperatures in the range of about 120° C. to about 180° C., thereby allowing the use of glass-lined or fluoropolymer-lined equipment. However, Co(II), Ni(II), Mn(II), V(II), Cr(II) or other transition-metals, which form oxidizable bromide salts, may also be used in the process. Alternatively, alkaline-earth metals which also form oxidizable bromide salts, such as Cu(II) or Mg(II) may be used. Hydrogen bromide is dissolved in the aqueous solution and neutralized by the metal hydroxide, metal oxide, metal oxy-bromide or mixtures of these species to yield metal bromide salt in solution and water which are removed from scrubber 38 via line 44. Any liquid higher molecular weight hydrocarbons condensed in scrubber 38 may be skimmed and withdrawn in line 37 and added to liquid higher molecular weight hydrocarbons exiting a product recovery unit 52 (i.e., the product recovery stage) and recovered as the desirable primary end products, i.e., hydrocarbon liquid products, in line 54.

The residual vapor phase, which contains olefins, higher molecular weight hydrocarbons or mixtures thereof, is removed from scrubber 38 as effluent and conveyed to a dehydrator 50 (i.e., the dehydration stage) via line 39 to remove substantially all water from the vapor stream via line 53. The dried vapor stream, which contains olefins, higher molecular weight hydrocarbons or mixtures thereof, is conveyed to product recovery unit 52 via line 51 where olefins, the C₅+ gasoline-range hydrocarbon fraction or mixtures thereof are recovered as hydrocarbon liquid products via line 54. Any conventional method of dehydration and liquids recovery, such as solid-bed desiccant adsorption followed by refrigerated condensation, cryogenic expansion, or circulating absorption oil or other solvent, as is used to process natural gas or refinery gas streams and/or to recover olefinic hydrocarbons within the purview of a skilled artisan, may be employed for this operation.

The residual gas from product recovery unit 52 which comprises the feed gas and essentially any other residual process gases remaining after the gas-liquid separation in the product recovery unit 52 is serially conveyed to the gas enrichment stage 422 and secondary dehydration stage 424 of the gas treatment sequence via line 62. The resultant methane-enriched gas output by the secondary dehydration stage 424 is compressed by compressor 58 and split into two fractions. A first fraction, which is equal to at least 2.5 times the inlet gas molar volume, is transported via line 62, combined with dry liquid bromine, conveyed by pump 24, heated in exchanger 26 to vaporize the bromine and fed into first reactor 30. The second fraction is drawn off of line 62 via line 63 which is regulated by control valve 60 at a rate sufficient to dilute the alkyl bromide concentration to second reactor 34 and absorb the heat of reaction. As such, second reactor 34 is maintained at the selected operating temperature, preferably in the range of about 300° C. to about 450° C., which maximizes conversion versus selectivity and minimizes the rate of catalyst deactivation due to the deposition of carbon. In sum, the dilution provided by the methane-enriched gas permits controlled selectivity of bromination in first reactor 30 and controlled moderation of the temperature in second reactor 34.

Water containing metal bromide salt in solution, which is removed from scrubber 38 via line 44, is passed to hydrocarbon stripper 47 wherein residual dissolved hydrocarbons are stripped from this aqueous phase by contact with incoming feed gas transported via line 11. The stripped aqueous solution is transported from hydrocarbon stripper 47 via line 65, cooled to a temperature in the range of about 0° C. to about 70° C. in heat exchanger 46 and passed to absorber 48 wherein residual bromine is recovered from vent stream in line 67. The aqueous solution effluent from adsorber 48 is transported via line 49 to a heat exchanger 40, preheated to a temperature in the range of about 100° C. to about 600° C., and most preferably in the range of about 120° C. to about 180° C., and passed to a third reactor 16 (i.e., the hydrogen bromide oxidation stage).

Oxygen or air is delivered to a bromine stripper 14 via line 10 by blower or compressor 13 at a pressure in the range of about ambient to about 5 bar to strip residual bromine from water. Water is removed from stripper 14 in line 64 and combined with water stream 53 from dehydrator 50 to form water effluent stream in line 56 which is removed from the process. The oxygen or air leaving bromine stripper 14 is fed via line 15 to third reactor 16 which operates at a pressure in the range of about ambient to about 5 bar and at a temperature in the range of about 100° C. to about 600° C., but most preferably in the range of about 120° C. to about 180° C. The oxygen or air oxidizes an aqueous metal bromide salt solution in third reactor 16 which yields elemental bromine and metal hydroxide, metal oxide, metal oxy-bromide or mixtures of these species. As stated above, although Co(II), Ni(II), Mn(II), V(II), Cr(II) or other transition-metals which form oxidizable bromide salts can be used, the preferred metal of the bromide salt is Fe(III), Cu(II), or Zn(II), or mixtures thereof. These are less expensive and readily oxidize at lower temperatures in the range of about 120° C. to about 180° C., which should allow the use of glass-lined or fluoropolymer-lined equipment. Alternatively alkaline-earth metals which also form oxidizable bromide salts, such as Ca(II) or Mg(II), could be used.

Hydrogen bromide reacts with the metal hydroxide, metal oxide, metal oxy-bromide or mixtures of these species so formed to once again yield the metal bromide salt and water. Heat exchanger 18 in third reactor 16 supplies heat to vaporize water and bromine. Thus, it is believed that the overall reactions result in the net oxidation of hydrogen bromide produced in first reactor 30 and second reactor 34 to elemental bromine and steam in the liquid phase. The reactions are catalyzed by the metal bromide/metal oxide or metal hydroxide operating in a catalytic cycle.

In the case where the metal bromide is Fe(III)Br₃, the reactions are believed to be:

Fe(+3a)+6Br(−a)+3H(+a)+3/2O₂(g)=3Br₂(g)+Fe(OH)₃ 1)

3HBr(g)+H₂O=3H(+a)+3Br(−a)+H₂O 2)

3H(+a)+3Br(−a)+Fe(OH)₃═Fe(+3a)+3Br(−a)+3H₂O 3)

In the case where the metal bromide is CU(II)Br₂, the reactions are believed to be:

4Cu(+2a)+8Br(−a)+3H₂0+3/2O₂(g)=3Br₂(g)+CuBr₂.3Cu(OH)₂ 1)

6HBr(g)+H₂O=6H(+a)+6Br(−a)+H₂O 2)

6H(+a)+6Br(−a)+CuBr₂.3Cu(OH)₂=4Cu(+2a)+8Br(−a)+6H₂O 3)

The elemental bromine and water and any residual oxygen (and/or nitrogen if air is utilized as the oxidant) leaving as vapor from the outlet of third reactor 16 via line 19 are cooled in condenser 20 at a temperature in the range of about 0° C. to about 70° C. and a pressure in the range of about ambient to 5 bar to condense the bromine and water and passed to three-phase separator 22. Since liquid water has a limited solubility for bromine, on the order of about 3% by weight, any additional bromine which is condensed forms a separate, denser liquid bromine phase in three-phase separator 22. The liquid bromine phase, however, has a notably lower solubility for water, on the order of less than 0.1%. Thus, a substantially dry bromine vapor can be easily obtained by condensing liquid bromine and water, decanting the water by simple physical separation and subsequently re-vaporizing liquid bromine.

Liquid bromine is pumped in line 25 from three-phase separator 22 via pump 24 to a pressure sufficient to mix with the methane-enriched gas in line 62. Thus, bromine is recovered and recycled within the process. The residual oxygen or nitrogen and any residual bromine vapor which is not condensed exits three-phase separator 22 and is passed via line 23 to bromine scrubber 48, wherein residual bromine is recovered by solution into and by reaction with reduced metal bromides in the aqueous metal bromide solution stream 65. Water is removed from separator 22 via line 27 and introduced into stripper 14.

Referring to FIG. 5, another embodiment of the gas-to-liquids conversion process of the present invention is shown wherein the gas treatment sequence of FIG. 1 is integrated with an alternate specific embodiment of a gas conversion sequence. As such, line 111 of FIG. 5 corresponds to the feed gas input in FIG. 1 and line 162 of FIG. 5 and associated process units correspond to the gas treatment sequence in FIG. 1. Furthermore, process units 130, 134, 138, 117, 150 and 152 of FIG. 5 correspond to stages 410, 412, 414, 416 and 418, respectively, of FIG. 1. The remaining elements shown in the embodiment of FIG. 5 which are common to the embodiment of FIG. 1 are designated in FIG. 5 by the same reference characters as used in FIG. 1.

In accordance with the embodiment of FIG. 5, a fresh feed gas having a substantially similar composition as the feed gas described above with reference to FIG. 1 is directed via line 111 to a hydrocarbon stripper 147 described in greater detail hereafter. A methane-enriched gas likewise having a substantially similar composition as the methane-enriched gas described above with reference to FIG. 1 exits the compressor 158 in line 162 downstream of the secondary dehydration stage 424 at a pressure in the range of about 1 bar to about 30 bar and is directed into line 125 where it mixes with dry liquid bromine being transported via line 125 by pump 124. The methane-enriched gas and dry liquid bromine pass through heat exchanger 126 wherein the liquid bromine is vaporized to dry bromine vapor. The resulting mixture of methane-enriched gas and dry bromine vapor is fed to a first reactor 130 (i.e., the alkane bromination stage). The molar ratio of lower molecular weight alkanes to dry bromine vapor in the mixture introduced into first reactor 130 is preferably in excess of 2.5:1. First reactor 130 has an inlet pre-heater zone 128 which heats the mixture to a reaction initiation temperature in the range of about 250° C. to about 400° C.

The lower molecular weight alkanes react exothermically with the dry bromine vapor in first reactor 130 at a relatively low temperature in the range of about 250° C. to about 600° C. and at a pressure in the range of about 1 bar to about 30 bar to produce gaseous alkyl bromides and hydrogen bromide vapors. The upper limit of the operating temperature range in first reactor 130 is greater than the upper limit of the reaction initiation temperature range due to the exothermic nature of the bromination reaction. In the case where the lower molecular weight alkane is methane, methyl bromide is formed in accordance with the following general reaction:

CH₄(g)+Br₂(g)→CH₃Br(g)+HBr(g)

This reaction occurs with a significantly high degree of selectivity to methyl bromide. Furthermore, selectivity to the mono-halogenated methyl bromide increases using a methane to bromine ratio of about 4.5:1. Small amounts of dibromomethane and tribromomethane are also formed in the bromination reaction. Higher alkanes, such as ethane, propane and butane, are also readily brominated resulting in mono and multiple brominated species such as ethyl bromides, propyl bromides and butyl bromides. If an alkane to bromine ratio of significantly less than about 2.5 to 1 is utilized, a lower selectivity to methyl bromide occurs and significant formation of undesirable carbon soot is observed.

The dry bromine vapor that is fed into first reactor 130 is preferably substantially water-free. It has been discovered that elimination of substantially all water vapor from the bromination step in first reactor 130 substantially eliminates the formation of unwanted carbon dioxide, thereby increasing the selectivity of alkane bromination to alkyl bromides and eliminating the large amount of waste heat generated in the formation of carbon dioxide from alkanes.

The effluent from first reactor 130, which contains alkyl bromides and hydrogen bromide, is withdrawn via line 131 and partially cooled in heat exchanger 132 before being conveyed to a second reactor 134 (i.e., the alkyl bromide conversion stage). The temperature to which the effluent is partially cooled in heat exchanger 132 is in the range of about 150° C. to about 350° C. when it is desired to convert the alkyl bromides to higher molecular weight hydrocarbons in second reactor 134 or in the range of about 150° C. to about 450° C. when it is desired to convert the alkyl bromides to olefins in second reactor 134. The alkyl bromides are reacted exothermically in second reactor 134 over a fixed bed 133 of crystalline alumino-silicate catalyst. The temperature and pressure employed in second reactor 134, as well as the crystalline alumino-silicate catalyst, determine the actual product(s) formed in second reactor 134.

The crystalline alumino-silicate catalyst in fixed bed 133 is preferably a zeolite catalyst and most preferably a ZSM-5 zeolite catalyst when it is desired to form higher molecular weight hydrocarbons. Although the zeolite catalyst is preferably in the hydrogen, sodium or magnesium form, the zeolite may also be modified by ion exchange with other alkali metal cations, such as Li, Na, K or Cs, with alkali-earth metal cations, such as Mg, Ca, Sr or Ba, or with transition metal cations, such as Ni, Mn, V, W, or to the hydrogen form. Other zeolite catalysts having varying pore sizes and acidities, which are synthesized by varying the alumina-to-silica ratio may be used in second reactor 134 as will be evident to a skilled artisan.

When it is desired to form olefins from the reaction of alkyl bromides in second reactor 134, the crystalline alumino-silicate catalyst employed in second reactor 134 is preferably a zeolite catalyst, and most preferably an X type or Y type zeolite catalyst. A preferred zeolite is 10X or Y type zeolite, although other zeolites with differing pore sizes and acidities, which are synthesized by varying the alumina-to-silica ratio may be used in the process as will be evident to a skilled artisan. Although the zeolite catalyst is preferably used in a protonic form, a sodium form or a mixed protonic/sodium form, the zeolite may also be modified by ion exchange with other alkali metal cations, such as Li, K or Cs, with alkali-earth metal cations, such as Mg, Ca, Sr or Ba, or with transition metal cations, such as Ni, Mn, V, W, or to the hydrogen form. These various alternative cations have an effect of shifting reaction selectivity. Other zeolite catalysts having varying pore sizes and acidities, which are synthesized by varying the alumina-to-silica ratio, may be used in second reactor 134 as will be evident to a skilled artisan.

The temperature at which second reactor 134 is operated is an important parameter in determining the selectivity of the reaction to higher molecular weight hydrocarbons or to olefins. Where a catalyst is selected to form higher molecular weight hydrocarbons in second reactor 134, it is preferred to operate second reactor 134 at a temperature within the range of about 150° to 450° C. Temperatures above about 300° C. in second reactor 134 result in increased yields of light hydrocarbons, such as undesirable methane, whereas lower temperatures increase yields of higher molecular weight hydrocarbon products. At the low end of the temperature range, for example, with methyl bromide reacting over ZSM-5 zeolite at temperatures as low as 150° C., methyl bromide conversion on the order of 20% is noted with a high selectivity toward C₅+ products. When the alkyl bromide reaction is carried out over the preferred zeolite ZSM-5 catalyst, cyclization reactions also occur such that C₇+ fractions are composed primarily of substituted aromatics.

At increasing temperatures approaching 300° C., methyl bromide conversion increases towards 90% or greater. However, selectivity towards C₅+ products decreases and selectivity towards lighter products, particularly undesirable methane, increases. Surprisingly very little ethane or C₂-C₃ olefin constituents are formed. At temperatures approaching 450° C. almost complete conversion of methyl bromide to methane occurs.

In the optimum operating temperature range between about 300° C. and 400° C., a small amount of carbon will build up on the catalyst over time during operation as a byproduct of the reaction, which causes a decline in catalyst activity over a range of hours, up to hundreds of hours, depending on the reaction conditions and the composition of the inlet gas. It is believed that higher reaction temperatures above about 400° C. associated with the formation of methane favor the thermal cracking of alkyl bromides and formation of carbon or coke and, hence, an increase in the rate of deactivation of the catalyst. Conversely, temperatures at the lower end of the range, particularly below about 300° C., may also contribute to coking due to a reduced rate of desorption of heavier products from the catalyst. Hence, operating temperatures within the range of about 150° C. to about 450° C., but preferably in the range of about 300° C. to about 400° C. in second reactor 134 balance increased selectivity of the desired C₅+ products and lower rates of deactivation due to carbon formation against higher conversion per pass, which minimizes the quantity of catalyst, recycle rates and equipment size required.

Where a catalyst is selected to form olefins in second reactor 134, it is preferred to operate second reactor 134 at a temperature within the range of about 250° C. to 500° C. Temperatures above about 450° C. in second reactor 134 can result in increased yields of light hydrocarbons, such as undesirable methane, and also deposition of coke, whereas lower temperatures increase yields of ethylene, propylene, butylene and higher molecular weight hydrocarbon products. When the alkyl bromide reaction is carried out over the preferred 10X zeolite catalyst, it is believed that cyclization reactions also occur such that C₇+ fractions contain substantial substituted aromatics.

At increasing temperatures approaching 400° C., it is believed that methyl bromide conversion increases towards 90% or greater. However, selectivity towards C₅+ products decreases and selectivity towards lighter products, particularly olefins, increases. At temperatures exceeding 550° C., it is believed that a high conversion of methyl bromide to methane and carbonaceous coke occurs.

In the preferred operating temperature range between about 300° C. and 450° C., a lesser amount of coke will likely build up on the catalyst over time during operation as a byproduct of the reaction. It is believed that higher reaction temperatures above about 400° C., associated with the formation of methane, favor the thermal cracking of alkyl bromides and formation of carbon or coke and, hence, an increase in the rate of deactivation of the catalyst. Conversely, temperatures at the lower end of the range, particularly below about 300° C., may also contribute to coking due to a reduced rate of desorption of heavier products from the catalyst. Hence, operating temperatures within the range of about 250° C. to about 500° C. in second reactor 134, but preferably in the range of about 300° C. to about 450° C. balance increased selectivity of the desired olefins and C₅+ products and lower rates of deactivation due to carbon formation against higher conversion per pass, which minimizes the quantity of catalyst, recycle rates and equipment size required.

The catalyst may be periodically regenerated in situ by isolating second reactor 134 from the normal process flow. Once isolated, second reactor 134 is purged with an inert gas via line 170 at a pressure in a range from about 1 to about 5 bar at an elevated temperature in the range of about 400° C. to about 650° C. to remove unreacted material adsorbed on the catalyst insofar as is practical. The deposited carbon is subsequently oxidized to CO₂ by addition of air or inert gas-diluted oxygen to second reactor 134 via line 170 at a pressure in the range of about 1 bar to about 5 bar at an elevated temperature in the range of about 400° C. to about 650° C. Carbon dioxide and residual air or inert gas are vented from second reactor 134 via line 175 during the regeneration period.

The effluent, which comprises hydrogen bromide and higher molecular weight hydrocarbons, olefins or mixtures thereof, is withdrawn from second reactor 134 via line 135, cooled in exchanger 36 to a temperature in the range of 0° C. to about 100° C. and combined with vapor effluent from hydrocarbon stripper 147 in line 112. The resulting mixture is passed to a scrubber 138 and contacted with a stripped recirculated water which has been transported to scrubber 138 via line 164 by any suitable means, such as pump 143, after the stripped recirculated water has been cooled in heat exchanger 155 to a temperature in the range of about 0° C. to about 50° C. Any liquid higher molecular weight hydrocarbons condensed in scrubber 138 may be skimmed, withdrawn as stream 137 and added to the hydrocarbon liquid products in line 154.

Hydrogen bromide is dissolved in the aqueous solution in scrubber 138, removed from scrubber 138 via line 144 and conveyed to hydrocarbon stripper 147. Residual hydrocarbons dissolved in the aqueous solution are stripped-out in hydrocarbon stripper 147 by contact with the incoming feed gas in line 111. The stripped aqueous phase from hydrocarbon stripper 147 is cooled in heat exchanger 146 to a temperature in the range of about 0° C. to about 50° C. and conveyed to absorber 148 via line 165 where residual bromine is recovered from vent stream 167.

The residual vapor phase, which contains olefins, higher molecular weight hydrocarbons or mixtures thereof, is removed from scrubber 138 as effluent and conveyed to a dehydrator 150 (i.e., the dehydration stage) via line 139 to remove substantially all water from the vapor stream via line 153. The dried vapor stream, which contains olefins, higher molecular weight hydrocarbons or mixtures thereof, is conveyed to a product recovery unit 152 (i.e., the product recovery stage) via line 151 to recover olefins, the C₅+ gasoline range hydrocarbon fraction or mixtures thereof as hydrocarbon liquid products in line 154. Any conventional method of dehydration and liquids recovery within the purview of a skilled artisan, such as solid-bed desiccant adsorption followed by refrigerated condensation, cryogenic expansion, or circulating absorption oil or other solvent, as is used to process natural gas or refinery gas streams and/or to recover olefinic hydrocarbons, may be employed for this operation.

The residual gas from product recovery unit 152 which comprises the feed gas and essentially any other residual process gases remaining after the gas-liquid separation in the product recovery unit 152 is serially conveyed to the gas enrichment stage 422 and secondary dehydration stage 424 of the gas treatment sequence via line 162. The resultant methane-enriched gas output by the secondary dehydration stage 424 is compressed by compressor 158 and split into two fractions. A first fraction, which is equal to at least 2.5 times the inlet gas molar volume, is transported via line 162, combined with dry liquid bromine, conveyed by pump 124, heated in exchanger 126 to vaporize the bromine and fed into first reactor 130. The second fraction is drawn off line 162 via line 163, which is regulated by control valve 160 at a rate sufficient to dilute the alkyl bromide concentration to second reactor 134 and absorb the heat of reaction. As such, second reactor 134 is maintained at the selected operating temperature, preferably in the range of about 300° C. to about 450° C., which maximizes conversion versus selectivity and minimizes the rate of catalyst deactivation due to the deposition of carbon. In sum, the dilution provided by the recycled vapor effluent permits controlled selectivity of bromination in first reactor 130 and controlled moderation of the temperature in second reactor 134.

Oxygen, oxygen-enriched air or air 110 is delivered to bromine stripper 114 via blower or compressor 113 at a pressure in the range of about ambient to about 5 bar and strips residual bromine from water. The stripped water is discharged from stripper 114 via line 164 and is divided into two portions. The first portion of stripped water is recycled to the process via line 164 while the second portion is removed from the process via line 156. The first portion of stripped water is cooled in heat exchanger 155 to a temperature in the range of about 20° C. to about 50° C. and maintained by any suitable means, such as pump 143, at a pressure sufficient to enter scrubber 138. The relative volume of the first portion is selected such that the hydrogen bromide solution effluent removed from scrubber 138 via line 144 has a concentration in the range from about 10% to about 50% by weight hydrogen bromide, and more preferably in the range of about 30% to about 48% by weight. This minimizes the amount of water which must be vaporized in exchanger 141 and preheater 119 and minimizes the vapor pressure of HBr over the resulting hydrogen bromide.

The dissolved hydrogen bromide in the aqueous solution effluent from adsorber 148 is transported via line 149 and combined with the oxygen, oxygen-enriched air or air leaving bromine stripper 114 via line 115. The combined aqueous solution effluent and oxygen, oxygen-enriched air or air is passed to a first side of heat exchanger 141, through preheater 119 where the mixture is preheated to a temperature in the range of about 100° C. to about 600° C., and most preferably in the range of about 120° C. to about 250° C., and on to a third reactor 117 (i.e., the hydrogen bromide oxidation stage) which is an oxidation reactor containing a metal bromide salt or metal oxide. The preferred metal of the bromide salt or metal oxide is Fe(III), Cu(II) or Zn(II), although Co(II), Ni(II), Mn(II), V(II), Cr(II) or other transition-metals which form oxidizable bromide salts can be used. Alternatively, alkaline-earth metals which also form oxidizable bromide salts, such as Ca (II) or Mg(II) could be used.

The metal bromide salt in third reactor 117 can be in the form of a concentrated aqueous solution, but preferably the concentrated aqueous salt solution is imbibed into a porous, high surface area, acid resistant inert support such as a silica gel. More preferably, the oxide form of the metal, which is in a concentration range of 10 to 20% by weight, is deposited on an inert support such as alumina with a specific surface area in the range of 50 to 200 m²/g.

Third reactor 117 operates at a pressure in the range of about ambient to about 5 bar and at a temperature in the range of about 100° C. to 600° C., and most preferably in the range of about 130° C. to 350° C. Within these operating ranges, the metal bromide is oxidized by oxygen, yielding elemental bromine and metal hydroxide, metal oxide or metal oxy-bromide species. Elemental bromine and metal oxides are yielded in the case of a supported metal bromide salt or in the case where third reactor 117 is operated at higher temperatures and lower pressures at which water primarily exists as a vapor. In any case, the hydrogen bromide reacts with the metal hydroxide, metal oxy-bromide or metal oxide species and is neutralized, restoring the metal It is believed that the overall reaction results in the net oxidation of hydrogen bromide produced in first reactor 130 and second reactor 134 to elemental bromine and steam. The reactions are catalyzed by the metal bromide/metal oxide or metal hydroxide operating in a catalytic cycle.

In the case where the metal bromide is Fe(III)Br₂ in an aqueous solution within a pressure and temperature range in which water may exist as a liquid, the reactions are believed to be:

Fe(+3a)+6Br(−a)+3H(+a)+3/2O₂(g)=3Br₂(g)+Fe(OH)3 1)

3HBr(g)+H₂O=3H(+a)+3Br(−a)+H₂O 2)

3H(+a)+3Br(−a)+Fe(OH)3=Fe(+3a)+3Br(−a)+3H₂O 3)

In the case where the metal bromide is CU(II)Br₂ in an aqueous solution and within a pressure and temperature range in which water may exist as a liquid, the reactions are believed to be:

4Cu(+2a)+8Br(−a)+3H₂0+3/2O₂(g)=3Br₂(g)+CuBr₂.3Cu(OH)₂ 1)

6HBr(g)+H₂O=6H(+a)+6Br(−a)+H₂O 2)

6H(+a)+6Br(−a)+CuBr₂.3Cu(OH)₂=4Cu(+2a)+8Br(−a)+6H₂O 3)

In the case where the metal bromide is Cu(II)Br₂ supported on an inert support and at higher temperature and lower pressure conditions at which water primarily exists as a vapor, the reactions are believed to be:

2Cu(II)Br₂=2Cu(I)Br+Br₂(g) 1)

2Cu(I)Br+O₂(g)=Br₂(g)+2Cu(II)O 2)

2HBr(g)+Cu(II)O═Cu(II)Br₂+H₂O(g) 3)

The elemental bromine and water and any residual oxygen (and/or nitrogen if air is utilized as the oxidant) leaving as vapor from the outlet of third reactor 117 are cooled in the second side of exchanger 141 and condenser 120 to a temperature in the range of about 0° C. to about 70° C. wherein the bromine and water are condensed and passed to three-phase separator 122. Since liquid water has a limited solubility for bromine, on the order of about 3% by weight, any additional bromine which is condensed forms a separate, denser liquid bromine phase in three-phase separator 122. The liquid bromine phase, however, has a notably lower solubility for water, on the order of less than 0.1%. Thus, a substantially dry bromine vapor can be easily obtained by condensing liquid bromine and water, decanting the water by simple physical separation and subsequently re-vaporizing liquid bromine. It is important to operate at conditions that result in the near complete reaction of hydrogen bromide so as to avoid significant residual hydrogen bromide in the condensed liquid bromine and water. Hydrogen bromide increases the miscibility of bromine in the aqueous phase, and at sufficiently high concentrations, results in a single ternary liquid phase.

Liquid bromine is pumped in line 125 from three-phase separator 122 via pump 124 to a pressure sufficient to mix with the methane-enriched gas in line 162. Thus the bromine is recovered and recycled within the process. The residual air, oxygen-enriched air or oxygen and any bromine vapor which is not condensed exits three-phase separator 122 and is passed via line 123 to bromine scrubber 148, wherein residual bromine is recovered by dissolution into the hydrogen bromide solution stream conveyed to scrubber 148 via line 165. Water is removed from the three-phase separator 122 via line 129 and passed to stripper 114.

The elemental bromine vapor and steam are condensed and easily separated in the liquid phase by simple physical separation yielding substantially dry bromine. The absence of significant water allows selective bromination of alkanes without production of CO₂ and the subsequent efficient and selective reactions of alkyl bromides to primarily C₂ to C₄ olefins, heavier products the C₅+ fraction of which contains substantial branched alkanes and substituted aromatics, or mixtures thereof. Byproduct hydrogen bromide vapor from the bromination reaction in first reactor 130 and the subsequent reaction in second reactor 134 is readily dissolved into an aqueous phase and neutralized by the metal hydroxide or metal oxide species resulting from oxidation of the metal bromide.

Although not shown, it is apparent to one of ordinary skill in the art that additional embodiments of the gas-to-liquids conversion process of the present invention can be readily configured, wherein the gas treatment sequences of FIGS. 2 and 3 can alternately be integrated with the specific embodiments of the gas conversion sequence of FIGS. 4 and 5. Nevertheless, it is understood that the present invention is not limited to any one specific gas conversion sequence. As such, additional alternate gas conversion sequences known in the art, such as the additional alternate gas conversion sequences disclosed in U.S. Pat. No. 7,348,464, which is incorporated herein by reference, may be adapted to the process of the present invention.

It is believed that the above-recited embodiments of the process for producing desirable hydrocarbon liquid products are less expensive than other conventional processes since the present process operates at low pressures in the range of about 1 bar to about 30 bar and at relatively low temperatures in the range of about 20° C. to about 600° C. for the gas phase and preferably about 20° C. to about 180° C. for the liquid phase. It is believed that these operating conditions permit the use of less expensive equipment of relatively simple design which are constructed from readily available metal alloys or glass-lined equipment for the gas phase and polymer-lined or glass-lined vessels, piping and pumps for the liquid phase.

It is believed that the present process for producing desirable hydrocarbon liquid products is also more efficient because less energy is required for operation and the production of excessive carbon dioxide as an unwanted byproduct is minimized. The process is capable of directly producing a mixed hydrocarbon product containing various molecular-weight constituents in the liquefied petroleum gas (LPG), olefin and motor gasoline fuels range that have substantial aromatic content, thereby significantly increasing the octane value of the gasoline-range fuel constituents.

While the foregoing preferred embodiments of the invention have been described and shown, it is understood that alternatives and modifications, such as those suggested and others, may be made thereto and fall within the scope of the present invention.

I claim:

1. A method for converting gaseous lower molecular weight alkanes to liquid higher molecular weight hydrocarbons comprising:
converting at least one C₂ to C₅ constituent contained in a feed gas to methane, thereby forming a methane-enriched gas from said feed gas, said methane-enriched gas having a methane fraction greater than a methane fraction of said feed gas;
reacting said methane-enriched gas and bromine to form alkyl bromide; and
reacting said alkyl bromide in the presence of a catalyst to form liquid higher molecular weight hydrocarbons and a residual gas.

2. The method of claim 1, wherein said feed gas contains methane.

3. The method of claim 1, wherein said feed gas contains methane and said methane fraction of said feed gas is greater than a C₂ to C₅ fraction of said feed gas.

4. The method of claim 1, wherein said at least one C₂ to C₅ constituent contained in said feed gas is converted to methane by pre-reforming said feed gas.

5. The method of claim 4, wherein said feed gas is pre-reformed by contacting said feed gas with an pre-former reactant in the presence of a pre-reforming catalyst.

6. The method of claim 1, wherein said methane-enriched gas has a methane fraction greater than about 50 mole %.

7. The method of claim 1, wherein said methane-enriched gas has a methane fraction of at least about 98 mole %.

8. The method of claim 4, wherein said feed gas is pre-reformed in a pre-reformer reactor, said method further comprising feeding at least a portion of said residual gas to said pre-reformer reactor and pre-reforming said residual gas therein.

9. A method for converting gaseous lower molecular weight alkanes to liquid higher molecular weight hydrocarbons comprising:
separating a C₃₊ fraction from a feed gas comprising lower molecular weight alkanes to form a C₃₊ product and an ethane-rich gas having an ethane fraction greater than an ethane fraction of said feed gas;
converting at least some of said ethane in said ethane-rich gas to methane, thereby forming a methane-enriched gas from said ethane-rich gas, said methane-enriched gas having a methane fraction greater than a methane fraction of said ethane-rich gas;
reacting said methane-enriched gas and bromine to form alkyl bromide; and
reacting said alkyl bromide in the presence of a catalyst to form liquid higher molecular weight hydrocarbons and a residual gas.

10. The method of claim 9, wherein said feed gas contains methane.

11. The method of claim 9, wherein said methane-enriched gas has a methane fraction of at least about 98 mole %.

12. The method of claim 9, wherein said ethane contained in said ethane-rich gas is converted to methane by pre-reforming said ethane-rich gas.

13. The method of claim 12, wherein said ethane-rich gas is pre-reformed in a pre-reformer reactor, said method further comprising feeding at least a portion of said residual gas to said pre-reformer reactor and pre-reforming said residual gas therein.

14. The method of claim 9, wherein said C₃₊ fraction is separated from said feed gas in a feed gas separator, said method further comprising recycling at least a portion of said residual gas to said feed gas separator.

15. The method of claim 12, wherein said feed gas is pre-reformed by contacting said feed gas with an pre-former reactant in the presence of a pre-reforming catalyst.

16. A method for converting gaseous lower molecular weight alkanes to liquid higher molecular weight hydrocarbons comprising:
separating a C₂₊ fraction from a feed gas comprising lower molecular weight alkanes to form an ethane-rich gas and a first methane-enriched gas, wherein said first methane-enriched gas has a methane fraction greater than a methane fraction of said feed gas and said ethane-rich gas has an ethane fraction greater than an ethane fraction of said feed gas;
converting at least some said ethane in said ethane-rich gas to methane, thereby forming a second methane-enriched gas having a methane fraction greater than a methane fraction of said ethane-rich gas;
reacting said first and second methane-enriched gases and bromine to form alkyl bromide; and
reacting said alkyl bromide in the presence of a catalyst to form liquid higher molecular weight hydrocarbons and a residual gas.

17. The method of claim 16, further comprising separating carbon dioxide from said second methane-enriched gas before reacting said second methane-enriched gas and bromine.

18. The method of claim 16, wherein said methane fraction of each of said first and second methane-enriched gases is at least about 98 mole %.

19. The method of claim 16, wherein said ethane contained in said ethane-rich gas is converted to methane by pre-reforming said ethane-rich gas.

20. The method of claim 19, wherein said ethane-rich gas is pre-reformed in a pre-reformer reactor, said method further comprising feeding at least a portion of said residual gas to said pre-reformer reactor and pre-reforming said residual gas therein.

21. The method of claim 16, wherein said C₂₊ fraction is separated from said feed gas in a feed gas separator, said method further comprising recycling a portion of said residual gas to said feed gas separator.

22. The method of claim 19, wherein said ethane-rich gas is pre-reformed by contacting said ethane-rich gas with a pre-former reactant in the presence of a pre-reforming catalyst.
